(19) United States
(12) Patent Application Publication
(10) Pub. No.: US 2026/0151200 A1
(43) Pub. Date: Jun. 4, 2026

(54) SENSOR-GUIDED ROBOTIC SURGERY

(71) Applicant: Covidien LP, Mansfield, MA (US)

(72) Inventors: Vikas Kaul, Plymouth, MN (US); Mark L. Palmer, Oak Grove, MN (US); Markus W. Reiterer, Plymouth, MN (US); Carl A. Schu, Plymouth, MN (US); Shawn C. Kelley, Ridgeland, WI (US); Michael A. Zemlok, Prospect, CT (US); Jonathan D. Thomas, New Haven, CT (US)

(21) Appl. No.: 19/122,863

(22) PCT Filed: Oct. 20, 2023
(86) PCT No.: PCT/IB2023/060627

§ 371 Date: Apr. 21, 2025

Related U.S. Application Data
(60) Provisional application No. 63/420,260, filed on Oct. 28, 2022.
(60) Provisional application No. 63/462,566, filed on Apr. 28, 2023.

(51) Int. Cl. A61B 34/32; A61B 5/00; A61B 34/00; A61B 34/20; A61B 34/35; A61B 90/00; A61L 31/14

(52) U.S. Cl. CPC A61B 34/32; A61B 5/6861; A61B 34/35; A61B 34/74; A61B 34/76; A61B 90/361; A61L 31/148; A61B 2034/2072; A61B 2562/12

(57) ABSTRACT

A surgical robotic system includes a robotic arm having a surgical instrument. The surgical robotic system also includes a surgeon console including a pair of hand controllers for controlling the robotic arm and the surgical instrument. The system further includes a sensor, which may be 3D printed, disposed on tissue and configured to measure at least one parameter pertaining to the tissue. The system additionally includes a controller configured to receive sensor data from the sensor, the sensor data including the at least one parameter and control at least one of the robotic arm or the surgical instrument based on the sensor data.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. There is a need for a system to receive different types of sensor data about the surgical site to make the system safer and automate certain processes.

### SUMMARY

According to one embodiment of the present disclosure, a surgical robotic system is disclosed. The surgical robotic system includes a robotic arm having a surgical instrument. The surgical robotic system also includes a surgeon console including a pair of hand controllers for controlling the robotic arm and the surgical instrument. The system further includes a sensor disposed on tissue and configured to measure at least one parameter pertaining to the tissue. The system additionally includes a controller configured to receive sensor data from the sensor, the sensor data including the at least one parameter, and to control at least one of the robotic arm or the surgical instrument based on the sensor data.

Implementations of the above embodiment may include one or more of the following features. According to one aspect of the above embodiment, the sensor may include at least one of an accelerometer, a strain gauge, a temperature sensor, a position tracker, or an impedance sensor. The at least one parameter may include at least one of motion, strain, temperature, position, or impedance. In embodiments, the sensor is also configured to sense chemical and physiological properties, such as pH and oxygenation. The sensor may be also configured to detect chemical markers, such as a fluorescent compound, to identify tumors and their boundaries. The detection of the molecule could help identify the extent and/or margins of the tumor for better removal. The sensor may output an optical signal detected by the stereoscopic camera with suitable illumination and filters.

The surgeon console may further include a display configured to output the sensor data. At least a portion of the sensor may be 3D printed. The controller may be further configured to provide haptic feedback to the pair of hand controllers based on the sensor data. In embodiments, feedback may be audio and/or visual.

According to another embodiment of the present disclosure, a method for controlling a surgical robotic system is disclosed. The method includes establishing a wireless connection between a sensor and a controller. The sensor is disposed on tissue and is configured to measure at least one parameter pertaining to the tissue. The method also includes transmitting sensor data from the sensor to the controller, the sensor data including the at least one parameter. The method also includes controlling a robotic arm including a surgical instrument based on the sensor data.

Implementations of the above embodiment may include one or more of the following features. According to one aspect of the above embodiment, the method may further include 3D printing at least a portion of the sensor. The sensor may include at least one of an accelerometer, a strain gauge, a temperature sensor, a position tracker, or an impedance sensor. The at least one parameter may include at least one of motion, strain, temperature, position, or impedance. The method may also include outputting the sensor data on a display of a surgeon console. The method may further include providing haptic feedback to a pair of hand controllers of the surgeon console based on the sensor data.

According to a further aspect of the above embodiment, a surgical robotic system is disclosed. The system includes a robotic arm having a surgical instrument and a surgeon console having a pair of hand controllers for controlling the robotic arm and the surgical instrument. The system also includes a plurality of implantable sensors disposed in tissue and configured to measure at least one parameter pertaining to the tissue. The system also includes a camera configured to capture a video feed of the surgical instrument and a controller configured to receive location data of the plurality of implantable sensors and generate a plurality of virtual markers based on the location data representing the plurality of implantable sensors. The system also includes a display configured to display the video feed with the plurality of markers. Implementations of the above embodiment may include one or more of the following features. According to one aspect of the above embodiment, each implantable sensor of the plurality of implantable sensors may include a substrate and at least one electronic component. The substrate may be formed from a biodegradable polymer. The electronic component may be formed from a biodegradable metal. The biodegradable metal may be formed from magnesium, zinc, iron, silicone, manganese, and alloys thereof. One implantable sensor of the plurality of implantable sensors may have a capsule shape and may be injectable into the tissue. One implantable sensor of the plurality of implantable sensors may have a disc shape.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a schematic diagram of a surgical site having a plurality of sensors disposed thereon; and

FIG. 7 is a flow chart of a method of controlling the surgical robotic system of FIG. 1 based on the sensors of FIG. 6 according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The inputs are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate a torque command for activating one or more actuators of the robotic arm, which would, in turn, move the robotic arm in response to the movement commands.

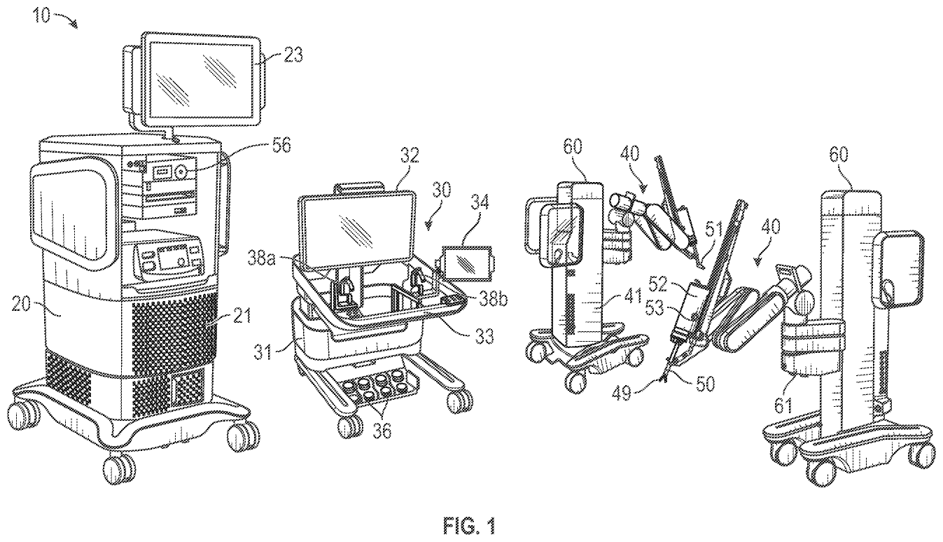

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to an image processing device 56, which may be disposed within the control tower 20. The image processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

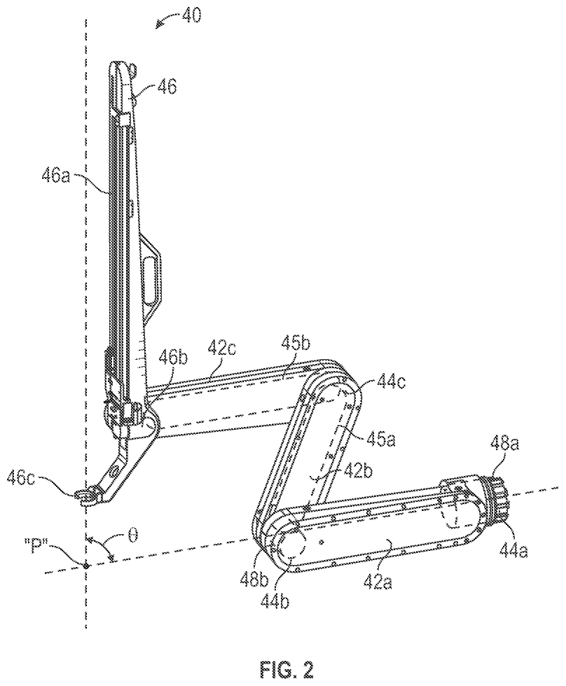

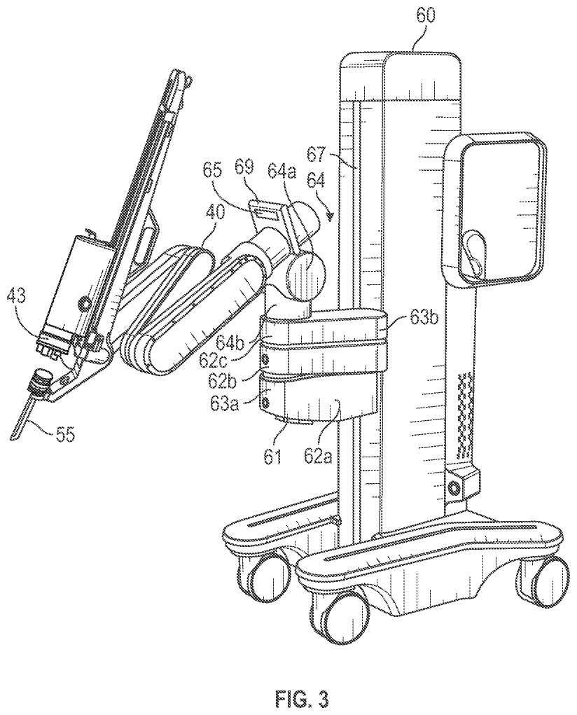

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

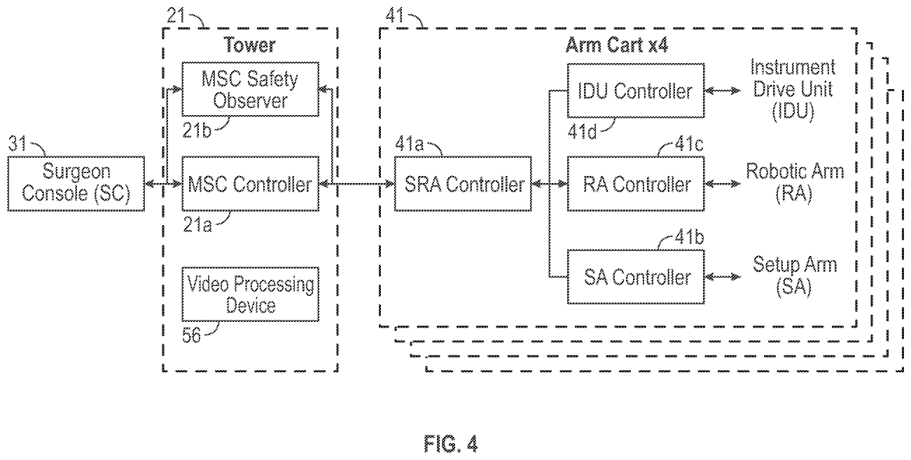

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a. Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

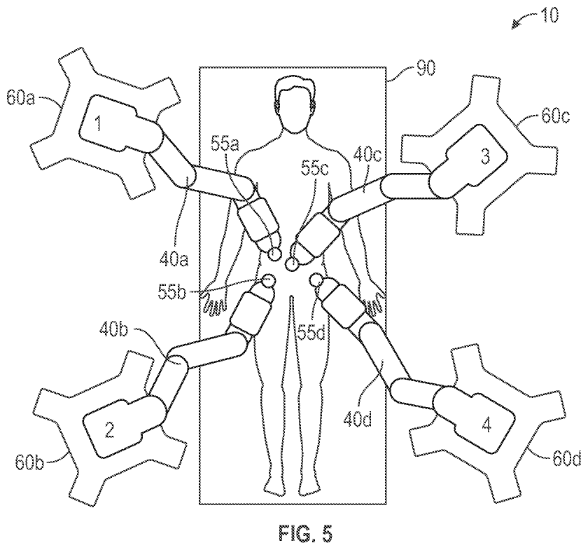

With reference to FIG. 5, the surgical robotic system 10 is set up around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52. The SIM 43 is also configured to secure a sterile drape (not shown) to the IDU 52.

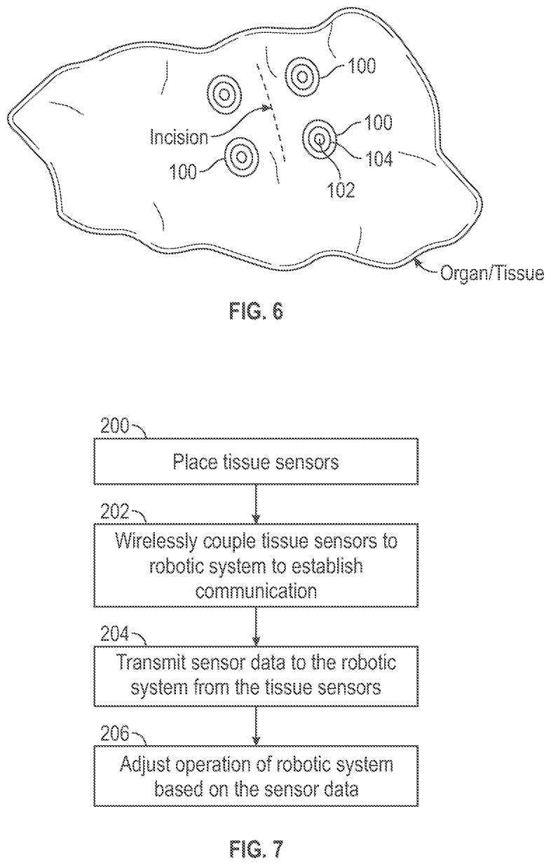

With reference to FIG. 6, the system 10 is configured to operate with one or more sensors 100 that are placed on the tissue and/or at the surgical site. The sensors 100 enhance a physician's awareness of the surgical site beyond the visual feedback from the camera 51, thereby reducing risk, standardizing procedures, and improving outcomes for patients. The sensors 100 may include an electronic component 102 disposed in or on a substrate 104. The electronic component 102 may include a transducer, which may be any electronic device configured to output an electric sensor signal in response to external stimulus, e.g., temperature, humidity, movement, strain, bioelectrical signals, etc. The electronic component 102 may also include a transceiver configured to transmit sensor signals to the system 10 having corresponding communication devices coupled to the controllers of the system 10.

The sensors 100 may have a disc-like shape as shown in FIG. 5 or any other suitable shape, such as a capsule having a length from about 5 mm to about 15 mm and a diameter from about 0.5 mm to about 2 mm allowing for injection of the sensor 100 into tissue through an appropriately sized needle (e.g., 12 to 15 gauge) injector (not shown). The injector may be disposed on the robotic arm 40 or may be a handheld device.

The substrate 104 may be 3D formed from any suitable biocompatible and/or biodegradable polymer and the electronic components 102 may be embedded therein. The substrate 104 may be printed in situ either onto the surface of deformable tissues or organs using a 3D printer attachment (not shown) coupled to one of the robotic arms 40. In embodiments, the sensors 100 may be printed outside the surgical site and placed onto the tissue/organ after printing is completed. Electronic components 102, such as an RFID antenna, may also be 3D printed. The substrate 104 may be coupled to the tissue using a patch or adhesive, which may also be biodegradable.

As used herein, the term “biodegradable” in reference to a material refers to the property of the material being able to be absorbed by the body. In the present application, the terms “biodegradable,” “bioresorbable,” “bioerodable,” and “bioabsorbable” are used interchangeably and are intended to mean the characteristic according to which a material decomposes, or loses structural integrity under body conditions (e.g., enzymatic degradation or hydrolysis) or are broken down (physically or chemically) under physiologic conditions in the body, such that the degradation products are excretable or absorbable by the body after a given period of time. The time period may vary, from about one hour to about several months or more, depending on the chemical nature of the material. In embodiments, the material may not be completely absorbed, provided the non-absorbed material is acceptable for medical use.

Suitable biodegradable polymers include, but are not limited to, aliphatic polyesters, polyamides, polyamines, polyalkylene oxalates, poly(anhydrides), polyamidoesters, copoly(ether-esters), poly(carbonates) including tyrosine derived carbonates, poly(hydroxyalkanoates) such as poly(hydroxybutyric acid), poly(hydroxyvaleric acid), and poly(hydroxybutyrate), polyimide carbonates, poly(imino carbonates) such as such as poly (bisphenol A-iminocarbonate and the like), polyorthoesters, polyoxaesters including those containing amine groups, polyphosphazenes, poly (propylene fumarates), polyurethanes, polymer drugs such as polydiflunisol, polyaspirin, and protein therapeutics, biologically modified (e.g., protein, peptide) bioabsorbable polymers, and copolymers, block copolymers, homopolymers, blends, and combinations thereof.

Suitable aliphatic polyesters include, but are not limited to, polylactide, polylactide-co-glycolide, polylactide-polycaprolactone, homopolymers and copolymers of lactide (including lactic acid, D-, L- and meso lactide), glycolide (including glycolic acid), epsilon-caprolactone, p-dioxanone (1,4-dioxan-2-one), trimethylene carbonate (1,3-dioxan-2-one), alkyl derivatives of trimethylene carbonate, Δ-valerolactone, β-butyrolactone, γ-butyrolactone, ε-decalactone, hydroxy butyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, α, α diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one, and polymer blends and copolymers thereof.

The sensors 100 are configured to track surface motion of the tissue using accelerometers or any other suitable motion sensor (e.g., IMU). In further embodiments, sensors 100 may be configured to measure impedance of the tissue based on an interrogatory electrical signal supplied to the tissue. Impedance measurements may be used to provide insight into any sub-surface vasculature and help reduce the risk of cutting into those vessels. In addition, impedance monitoring may be used to measure edema build-up and provide electrical mapping of the depolarization activity of the heart, gastrointestinal tract, nerve root monitoring, etc.

The sensors 100 may include one or more strain gauges configured to measure strain imparted to tissue based on stretching, deformation, etc. The strain data may be used to provide haptic feedback to the surgeon console 30 and/or create a virtual model (e.g., digital twin) of the tissue. The sensors 100 may also include a temperature sensor. In embodiments, feedback may also be audio and/or visual messages delivered through the surgeon console 30.

In addition, the sensors 100 may include passive position markers or trackers that are interrogated by the system 10 to track position of the sensors 100. The position of the sensors 100 may be used by the system 10 to determine relative position of the instruments 50 and/or robotic arms 40 to the sensors 100, and by extension, position of the instruments 50 and/or robotic arms 40 relative to the surgical site. The position of the sensors 100 may be used to guide surgical robotic procedures by setting up virtual boundaries for the robotic arms 40 beyond which the robotic arms 40 may not be moved even when commanded by the user through the surgeon console 30. In addition to calculating position, a relative change of two or more sensors 100 may also be used to calculate strain using digital image correlation (DIC).

Various electronic components of the sensors 100 described above may be formed from biodegradable metal alloys including magnesium and zinc, such as commercially-available HP-Mg, FAsorbMg™, MGYREZr, and WE43, as well as iron and alloys of iron containing Mg, Mn, and Si.

With reference to FIG. 7, a method of using the sensors 100 with the system 10 is disclosed. At step 200 the sensors 100 are placed on the tissue, either by being printed directly on the tissue or by being printed outside the surgical site and placed on the tissue thereafter. After printing, communication between the sensors 100 and the system 10 is established at step 202. This may include pairing the sensors 100 to establish a wireless connection or interrogating the sensors 100. During the surgical procedure, at step 204, the sensors 100 continuously and/or periodically provide sensor data to the system 10 including the data described above, e.g., impedance, tissue strain, temperature, etc. The system 10 receives the sensor data and adjusts operation of one or more components based on the sensor data at step 206. Adjustments may include providing haptic feedback through the hand controllers 38a and 38b based on measured strain. The system 10 is further configured to detect tissue properties, (e.g., vasculature, edema build up, through impedance and/or temperature measurements, etc.), and output alerts, graphical overlays, and other information for the user at the surgeon console 30 or other displays of the system 10. In addition, the system 10 may also automatically control position of the robotic arms 40 and/or instrument 50 based on tracking data from the sensors 100, e.g., maintaining the robotic arms 40 within the virtual boundary.

The sensors 100 may also be used as tissue markers and may be injected or inserted into various portions of tissue or organ. The sensors 100 may be used as markers in triangulating the tissue. Location of the sensors 100 may be determined using X-rays, ultrasound waves, electromagnetic waves, etc. An imaging or locating device e.g., X-ray generator/detector, ultrasound probe, electromagnetic transceivers, etc. may be used to provide imaging and/or signal data to the image processing device 56, which may then overlay or superimpose location of the sensors 100 over the video feed provided by the camera 51. The sensors 100 may be displayed as virtual markers or any other geometric shape on the video feed shown on the display 32 allowing for localization of the tissue as well as the instruments 50 shown in the video feed.

The sensors 100 are formed from corresponding materials and/or include coatings made from suitable materials, e.g., radiopaque materials for X-ray triangulation, e.g., magnetic materials and/or metals for electromagnetic triangulation, ultrasound reflective materials for ultrasound imaging. In embodiments, biodegradable composites described above may be used such that the sensors 100 may be left behind in the tissue to degrade and be resorbed by the tissue. Ultrasound reflectivity of the sensors 100 may be provided by applying an echogenic coating such as those disclosed in U.S. Pat. No. 6,106,473, titled “Echogenic Coatings”, filed on Nov. 6, 1997, the entire disclosure of which is incorporated by reference herein. For electromagnetic tracking, the sensors 100 may include an antenna, a coil, or any other suitable structure configured to receive and transmit, i.e., passively, electromagnetic signals suitable for triangulation of the tissue markers. Suitable metals for forming electromagnetic markers include biodegradable metals, such as magnesium, zinc, and alloys thereof.

For X-ray triangulation, the sensors 100 may have a radiopaque material contained in the sensors 100, i.e., as a pellet, sphere, etc., or as an additive the substrate 104. In any one or more embodiments above, the radiopaque material may be a metal, such as platinum, iridium, cobalt chromium alloy, stainless steel (316L), high nitrogen stainless steel, Nitinol, tantalum, nickel, tungsten, titanium, gold, magnesium, or an alloy or combination of any two or more thereof. In any one or more embodiments, the radiopaque material may be biodegradable. In embodiments, radiopaque additives, such as barium sulfate, may be added to the biodegradable polymer of the sensors 100 or applied as a coating thereto.

The sensors 100 may also be used to monitor implants, e.g., hernia meshes. Ventral or inguinal hernia meshes contour to the patient's abdominal wall planes. The sensors 100 may be attached to or otherwise incorporated into the tissue implants. The sensors 100 may be used to monitor deformation and mechanical loads on the tissue implant via strain gauges. The sensors 100 could be used in placement and/or localizing of the tissue implant, which may be used to ensure the tissue implant is not placing tension on the repair (i.e., in tension free repairs).

In further embodiments, the sensors 100 may be attached to loading labels and/or implant tackers. Intraoperatively, the labels/sensors could provide the surgeon direction as to how far to space tacks apart from one another. Additionally, the labels/sensors could be used to ensure the repair is tension-free based on the strain/load measurements.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including a surgical instrument;
a surgeon console including a pair of hand controllers for controlling the robotic arm and the surgical instrument;
a sensor disposed on tissue and configured to measure at least one parameter pertaining to the tissue; and
a controller configured to:
receive sensor data from the sensor, the sensor data including the at least one parameter; and
control at least one of the robotic arm or the surgical instrument based on the sensor data.

2. The surgical robotic system according to claim 1, wherein the sensor includes at least one of an accelerometer, a strain gauge, a temperature sensor, a position tracker, or an impedance sensor.

3. The surgical robotic system according to claim 1, wherein the at least one parameter includes at least one of motion, strain, temperature, position, or impedance.

4. The surgical robotic system according to claim 1, wherein the surgeon console includes a display configured to output the sensor data.

5. The surgical robotic system according to claim 1, wherein at least a portion of the sensor is 3D printed.

6. The surgical robotic system according to claim 1, wherein the controller is further configured to provide haptic feedback to the pair of hand controllers based on the sensor data.

7. A method for controlling a surgical robotic system, the method comprising:
establishing a wireless connection between a sensor and a controller, the sensor being disposed on tissue and configured to measure at least one parameter pertaining to the tissue;
transmitting sensor data from the sensor to the controller, the sensor data including the at least one parameter; and
controlling a robotic arm including a surgical instrument based on the sensor data.

8. The method according to claim 7, further comprising:
3D printing at least portion of the sensor.

9. The method according to claim 7, wherein establishing the wireless connection includes establishing a wireless connection between the controller and at least one of an accelerometer, a strain gauge, a temperature sensor, a position tracker, or an impedance sensor.

10. The method according to claim 7, wherein transmitting sensor data includes transmitting at least one of motion, strain, temperature, position, or impedance of the tissue.

11. The method according to claim 7, further comprising:
outputting the sensor data on a display of a surgeon console.

12. The method according to claim 11, further comprising:
providing haptic feedback to a pair of hand controllers of the surgeon console based on the sensor data.

13. A surgical robotic system comprising:
a robotic arm including a surgical instrument;
a surgeon console including a pair of hand controllers for controlling the robotic arm and the surgical instrument;
a plurality of implantable sensors disposed in tissue and configured to measure at least one parameter pertaining to the tissue;
a camera configured to capture a video feed of the surgical instrument; and
a controller configured to:
receive location data of the plurality of implantable sensors; and
generate a plurality of virtual markers based on the location data of the plurality of implantable sensors; and
a display configured to display the video feed with the plurality of markers.

14. The surgical robotic system according to claim 13, wherein each implantable sensor of the plurality of implantable sensors includes a substrate and at least one electronic component.

15. The surgical robotic system according to claim 14, wherein the substrate is formed from a biodegradable polymer.

16. The surgical robotic system according to claim 15, wherein the biodegradable polymer is selected from the group consisting of aliphatic polyesters, polyamides, polyamines, polyalkylene oxalates, poly(anhydrides), polyamidoesters, copoly(ether-esters), poly(carbonates) including tyrosine derived carbonates, poly(hydroxyalkanoates) such as poly(hydroxybutyric acid), poly(hydroxyvaleric acid), and poly(hydroxybutyrate), polyimide carbonates, poly(imino carbonates) such as such as poly (bisphenol A-iminocarbonate and the like), polyorthoesters, polyoxaesters including those containing amine groups, polyphosphazenes, poly (propylene fumarates), polyurethanes, polymer drugs such as polydiflunisol, polyaspirin, and protein therapeutics, biologically modified (e.g., protein, peptide) bioabsorbable polymers, and copolymers, block copolymers, homopolymers, blends, and combinations thereof.

17. The surgical robotic system according to claim 14, wherein the at least one electronic component is formed at least in part from a biodegradable metal.

18. The surgical robotic system according to claim 17, wherein the biodegradable metal is selected from the group consisting of magnesium, zinc, iron, silicone, manganese, and alloys thereof.

19. The surgical robotic system according to claim 14, wherein at least one implantable sensor of the plurality of implantable sensors has a capsule shape and is injectable into the tissue.

20. The surgical robotic system according to claim 14, wherein at least one implantable sensor of the plurality of implantable sensors has a disc shape.
